SEQUENCE LISTING (2 sequences)

SEQ ID NO: 1 - protein, 9 aa, Unknown
  Signature sequence of conserved catalytic domain of protein tyrosine phosphatases (PTPs)
  MISC_FEATURE (1)..(1): Xaa = Isoleucine or Valine
  MISC_FEATURE (4)..(5): Xaa = any amino acid
  MISC_FEATURE (7)..(8): Xaa = any amino acid
  MISC_FEATURE (9)..(9): Xaa = Serine or Threonine
XHCXXGXXX

SEQ ID NO: 2 - protein, 13 aa, Artificial Sequence
  Synthesized substrate peptide
  MOD_RES (7)..(7): PHOSPHORYLATION
TSTEPQYQPGENL

(19) United States
(12) Patent Application Publication
(10) Pub. No.: US 2007/0060551 A1
(43) Pub. Date: Mar. 15, 2007

(54) Methods of using isothiazole derivatives to treat cancer or inflammation

(75) Inventors: Zaihui Zhang, Vancouver (CA); Timothy S. Daynard, Vancouver (CA); Gabriel Bela Kalmar, Richomond (CA)

Correspondence Address: SEED INTELLECTUAL PROPERTY LAW GROUP PLLC, 701 FIFTH AVE, SUITE 5400, SEATTLE, WA, 98104, US

(73) Assignee: QLT, Inc., Vancouver, BC (CA)

(21) Appl. No.: 10/517,760

(22) PCT Filed: Jun. 11, 2003
(86) PCT No.: PCT/CA03/00864

§ 371 Date: Mar. 31, 2006

Related U.S. Application Data
(60) Provisional application No. 60/388,939, filed on Jun. 13, 2002.

(51) Int. Cl. A61K 31/655; A61K 31/425

(52) U.S. Cl. 514/149; 514/372

(57) ABSTRACT

Methods of using substituted 3,5-dithio-, disulfinyl-or disulfonyl-isothiazole derivatives to treat cancer or inflammation in a mammal and pharmaceutical compositions containing such derivatives are disclosed.

### FIELD OF THE INVENTION

This invention is directed to methods of using isothiazole derivatives.

### BACKGROUND OF THE INVENTION

Protein phosphorylation is a common regulatory mechanism used by cells to selectively modify proteins carrying regulatory signals from outside the cell to the nucleus. The proteins that execute these biochemical modifications are a group of enzymes known as protein kinases and protein phosphatases. They may further be defined by the substrate residue that they target for phosphorylation. Kinases and protein kinase pathways are involved in most cell signaling, and many of the pathways play a role in human disease. Protein tyrosine phosphorylation is an important mechanism for transmitting extracellular stimuli in biochemical and cellular events such as cell attachment, mitogenesis, differentiation and migration (see e.g., Li et al., Seminars in Immunology (2000), Vol. 12, pp. 75-84, and Neel et al., Current Opinion in Cell Biology (1997), Vol. 9, pp. 193-204).

Phosphorylation is important in signal transduction mediated by receptors via extracellular biological signals such as growth factors or hormones. For example, many oncogenes are kinases or phosphatases, i.e. enzymes that catalyze protein phosphorylation or dephosphorylation reactions or are specifically regulated by phosphorylation. In addition, a kinase or phosphatase can have its activity regulated by one or more distinct kinase or phosphatases, resulting in specific signaling cascades.

All protein tyrosine phosphatases (PTPs) have a conserved catalytic domain characterized by a signature sequence (IN)HCXXGXX(S/T). Biochemical and kinetic studies have demonstrated that the cysteine residue found in this signature sequence is essential for catalytic activity of PTPs since this mutation of this cysteine completely abolishes PTP activity. See, Flint, A. J., et al., Proceedings of the National Academy of Sciences of the United States of America 94 (1997), pp. 1680-1685.

### Description of the Related Art

PCT Published Patent Application, WO 99/61467 (McGill University), describes agents that interfere with the binding of PTPN12 (PTP-PEST) to domains of signalling proteins as inhibitors of cell migration and/or of focal adhesion.

U.S. Pat. No. 6,262,044 (Novo Nordisk) describes certain protein tyrosine phosphatase inhibitors and provides a detailed description of the discovery of protein tyrosine phosphatases and their pathophysiological roles.

### SUMMARY OF THE INVENTION

This invention is directed to the use of certain isothiazole derivatives in treating hyperproliferative disorders, e.g., cancer, inflammation, etc. in a mammal. Of particular interest are hyperproliferative disorders associated with cellular modulation of protein phosphorylation states, i.e. altered activity of phosphorylation modifying enzyme(s), e.g. protein tyrosine kinases and protein tyrosine phosphatases. In one aspect of the invention, compounds and pharmaceutical compositions of the invention are used to inhibit the activity of PTPN12. This enzyme has been associated with alterations in the phosphorylation state of cellular proteins.

Accordingly, in one aspect, this invention provides a method of treating cancer in a mammal, which method comprises administering to the mammal in need thereof a therapeutically effective amount of a compound of formula (I):

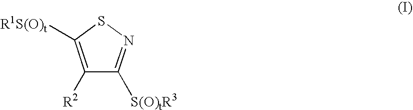

wherein:

each t is independently 0, 1 or 2;

R¹ and R³ are each independently alkyl, alkenyl, aryl, aralkyl, aralkenyl, cycloalkyl, cycloalkylalkyl, cycloalkylalkenyl, haloalkyl, haloalkenyl, haloalkoxyalkyl, haloalkoxyalkenyl, —R⁴—N═N—O—R⁵, —N(R⁶)₂ or heterocyclylalkyl;

R² is hydrogen, alkyl, alkenyl, aryl, aralkyl, aralkenyl, cycloalkyl, cycloalkylalkyl, cycloalkylalkenyl, halo, haloalkyl, haloalkenyl, cyano, nitro, —R⁴—N═N—O—R⁵, —OR⁶, —C(O)OR⁶, —N(R⁶)₂, —C(O)N(R⁶)₂, —N(R ⁶)C(O)OR⁵, —N(R⁶)C(O)N(R⁶)₂, heterocyclyl or heterocyclylalkyl;

R⁴ is a bond or a straight or branched alkylene or alkenylene chain;

each R⁵ is independently hydrogen, alkyl, alkenyl, haloalkyl, haloalkenyl, aralkyl, aralkenyl, cycloalkyl, cycloalkylalkyl or cycloalkylalkenyl; and

each R⁶ is independently hydrogen, alkyl, alkenyl, haloalkyl, haloalkenyl, aryl, aralkyl, aralkenyl, cycloalkyl, cycloalkylalkyl or cycloalkylalkenyl;

as a single stereoisomer, a mixture of stereoisomers, or as a racemic mixture of stereoisomers; or as a solvate or polymorph; or as a pharmaceutically acceptable salt thereof.

In another aspect, this invention provides a method of treating inflammation in a mammal, which method comprises administering to the mammal in need thereof a therapeutically effective amount of a compound of formula (I), as set forth above, as a single stereoisomer, a mixture of stereoisomers, or as a racemic mixture of stereoisomers; or as a solvate or polymorph; or as a pharmaceutically acceptable salt thereof.

In another aspect, this invention provides a method of treating hyperproliferative disorders in a mammal, which method comprises administering to the mammal in need thereof a therapeutically effective amount of a compound of formula (I), as set forth above, as a single stereoisomer, a mixture of stereoisomers, or as a racemic mixture of stereoisomers; or as a solvate or polymorph; or as a pharmaceutically acceptable salt thereof.

In another aspect, this invention provides a method of treating a mammal having a disorder or condition associated with hyperproliferation and tissue remodelling or repair, wherein said method comprises administering to the mammal having the disorder or condition a therapeutically effective amount of a compound of formula (I), as set forth above, as a single stereoisomer, a mixture of stereoisomers, or as a racemic mixture of stereoisomers; or as a solvate or polymorph; or as a pharmaceutically acceptable salt thereof.

In another aspect, this invention provides a method of treating a mammalian cell with a compound of formula (I), as set forth above, as a single stereoisomer, a mixture of stereoisomers, or as a racemic mixture of stereoisomers; or as a solvate or polymorph; or as a pharmaceutically acceptable salt thereof; wherein the method comprises administering the compound of formula (I) to a mammalian cell and the compound of formula (I) is capable of inhibiting the activity of PTPN12 within the mammalian cell.

In another aspect, this invention provides a pharmaceutical composition useful in treating cancer or inflammation in a human, wherein the pharmaceutical composition comprises a pharmaceutically acceptable carrier, diluent or excipient and a compound of formula (II):

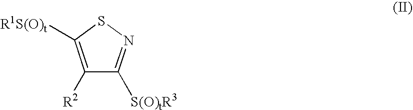

wherein:

each t is independently 0, 1 or 2;

R¹ and R³ are each independently alkyl, alkenyl, aryl, aralkyl, aralkenyl, cycloalkyl, cycloalkylalkyl, cycloalkylalkenyl, haloalkyl, haloalkenyl, haloalkoxyalkyl, haloalkoxyalkenyl, —R⁴—N═N—O—R⁵, —N(R⁶)₂ or heterocyclylalkyl;

R² is hydrogen, alkyl, alkenyl, aryl, aralkyl, aralkenyl, cycloalkyl, cycloalkylalkyl, cycloalkylalkenyl, halo, haloalkyl, haloalkenyl, nitro, —R⁴—N═N—O—R⁵, —OR⁶, —C(O)OR⁶, —N(R⁶)₂, —C(O)N(R⁶)₂, —N(R⁶)C(O)OR⁵, —N(R⁶)C(O)N(R⁶)₂, heterocyclyl or heterocyclylalkyl;

R⁴ is a bond or a straight or branched alkylene or alkenylene chain;

each R⁵ is independently hydrogen, alkyl, alkenyl, haloalkyl, haloalkenyl, aralkyl, aralkenyl, cycloalkyl, cycloalkylalkyl or cycloalkylalkenyl; and

each R⁶ is independently hydrogen, alkyl, alkenyl, haloalkyl, haloalkenyl, aryl, aralkyl, aralkenyl, cycloalkyl, cycloalkylalkyl or cycloalkylalkenyl;

provided that when t is 0 and R¹ and R³ are both methyl, R² can not be —C(O)OH, —C(O)NH₂, carboxymethyl or unsubstituted phenyl;

as a single stereoisomer, a mixture of stereoisomers, or as a racemic mixture of stereoisomers; or as a solvate or polymorph; or as a pharmaceutically acceptable salt thereof.

In another aspect, this invention provides compounds of formula (II) as set forth above.

### DETAILED DESCRIPTION OF THE INVENTION

Definitions

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art. As used in the specification and appended claims, unless specified to the contrary, the following terms have the meaning indicated.

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by one or more substituents selected from the group consisting of hydroxy, alkoxy, aryloxy, haloalkoxy, cyano, nitro, mercapto, alkylthio, —R⁴—N═N—O—R⁵, —N(R⁶)₂, —C(O)OR⁶, —C(O)N(R⁶)₂, —N(R⁶)C(O)OR⁵, —N(R⁶)C(O)N(R⁶)₂, S(O)ₜR⁶ (where t is 0 to 2) and S(O)ₜN(R⁶)₂ (where t is 0 to 2)where each R⁴, R⁵ and R⁶ are as defined above in the Summary of the Invention. Unless stated otherwise specifically in the specification, it is understood that for radicals, as defined below, that contain a substituted alkyl group that the substitution can occur on any carbon of the alkyl group.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by one or more substituents selected from the group consisting of hydroxy, alkoxy, aryloxy, haloalkoxy, cyano, nitro, mercapto, alkylthio, —R⁴—N═N—O—R⁵, —N(R⁶)₂, —C(O)OR⁶, —C(O)N(R⁶)₂, —N(R⁶)C(O)OR⁵, —N(R⁶)C(O)N(R⁶)₂, S(O)ₜR⁶ (where t is 0 to 2) and S(O)ₜN(R⁶)₂ (where t is 0 to 2) where each R⁴, R⁵ and R6 are as defined above in the Summary of the Invention. Unless stated otherwise specifically in the specification, it is understood that for radicals, as defined below, that contain a substituted alkenyl group that the substitution can occur on any carbon of the alkenyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of hydroxy, alkoxy, aryloxy, haloalkoxy, cyano, nitro, mercapto, alkylthio, cycloalkyl, —R⁴—N═N—O—R⁵, —N(R⁶)₂, —C(O)OR⁶, —C(O)N(R⁶)₂, —N(R⁶)C(O)OR⁵, —N(R⁶)C(O)N(R⁶)₂, S(O)ₜR⁶ (where t is 0 to 2) and S(O)ₜN(R⁶)₂ (where t is 0 to 2)where

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene” and “alkylene chain” refer to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —R⁴—N═N—O—R⁵, —N(R⁶)₂, —C(O)OR⁶, —C(O)N(R⁶)₂, —N(R⁶)C(O)OR⁵, —(R⁶)C(O)N(R⁶)₂, S(O)ₜR⁶ (where t is 0 to 2) and S(O)ₜN(R⁶)₂ (where t is 0 to 2) where each R⁴, R⁵ and R⁶ are as defined above in the Summary of the Invention. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —R⁴—N═N—O—R⁵, —N(R⁶)₂, —C(O)OR⁶, —C(O)N(R⁶)₂, —N(R⁶)C(O) OR⁵, —N(R⁶)C(O)N(R⁶)₂, S(O)ₜR⁶ (where t is 0 to 2) and S(O)ₜN(R⁶)₂ (where t is 0 to 2) where each R⁴, R⁵ and R⁶ are as defined above in the Summary of the Invention. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —R⁴—N═N—O—R⁵, —N(R⁶)₂, —C(O)OR⁶, —C(O)N(R⁶)₂, —N(R⁶)C(O)OR⁵, —N(R⁶)C(O)N(R⁶)₂, S(O)ₜR⁶ (where t is 0 to 2) and S(O)^{t}N(R⁶)₂ (where t is 0 to 2) where each R⁴, R⁵ and R⁶ are as defined above in the Summary of the Invention.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Cycloalkylalkenyl” refers to a radical of the formula —R_{f}R_{d} where R_{f} is an alkenyl radical as defined above and R_{d} is a cycloalkyl radical as defined above. The alkenyl radical and the cycloalkyl radical may be optionally substituted as defined above.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkenyl” refers to an alkenyl radical, as defined above, that is substituted by one or more halo radicals, as defined above, e.g., 2-ethenyl, 3-bromoprop-1-enyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Haloalkoxyalkyl” refers to an alkyl radical, as defined above, that is substituted by one or more haloalkoxy radicals, as defined above, e.g., trifluoromethoxymethyl, 2-(difluoromethoxy)ethyl, and the like.

“Haloalkoxyalkenyl” refers to an alkenyl radical, as defined above, that is substituted by one or more haloalkoxy radicals, as defined above, e.g., 2-(trifluoromethoxy)ethenyl, 3-(trichloromethoxy)prop-1-enyl, and the like.

“Heterocyclyl” refers to a stable 3- to 15-membered ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be aromatic or partially or fully saturated. The heterocyclyl radical may not be attached to the rest of the molecule at any heteroatom atom. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,5]imidazo[1,2-a]pyridinyl; carbazolyl, cinnolinyl, dioxolanyl, decahydroisoquinolyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyl” is meant to include heterocyclyl radicals as defined above which are optionally substituted by one or more substituents selected from the group consisting of alkyl, alkenyl, aryl, aralkyl, aralkenyl, cycloalkyl, cycloalkylalkyl, halo, haloalkyl, haloalkoxy, nitro, cyano, heterocyclyl, heterocyclylalkyl, —OR⁶₁, —R⁴—N═N—O—R⁵, —N(R⁶)₂, —C(O)OR⁶, —C(O)N (R⁶)₂, —N(R⁶)C(O)OR⁵, —N(R⁶)C(O)N(R⁶)₂, S(O)ₜR⁶ (where t is 0 to 2) and S(O)ₜN(R⁶)₂ (where t is 0 to 2) where each R⁴, R⁵ and R⁶ are as defined above in the Summary of the Invention.

“Heterocyclylalkyl” refers to a radical of the formula —RₐRₑ where Rₐ is an alkyl radical as defined above and Re is a heterocyclyl radical as defined above, and if the heterocyclyl is a nitrogen-containing heterocyclyl, the heterocyclyl may be attached to the alkyl radical at the nitrogen atom. The heterocyclyl radical may be optionally substituted as defined above.

As used herein, compounds which are “commercially available” may be obtained from standard commercial sources including Acros Organics (Pittsburgh, Pa.), Aldrich Chemical (Milwaukee Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah.), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, U.T.), Pfaltz & Bauer, Inc. (Waterbury, Conn.), Polyorganix (Houston, Tex.), Pierce Chemical Co. (Rockford, Ill.), Riedel de Haen AG (Hannover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified though various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandier et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th. Ed., Wiley Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalenty bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and domestic animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic add, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“PTPN12” refers to the Human Genome Organization (HUGO) Nomenclature Committee's name for protein tyrosine phosphatase, non-receptor like 12. PTPN12 is also known as PTP-PEST and PTPG1. The coding sequence may be accessed at Genbank; M93425;. and is disclosed by Yang et al. (1993) J. Biol. Chem. 268 (9), 6622-6628.

“Therapeutically effective amount” refers to that amount of a compound of formula (I) which, when administered to a mammal, preferably a human, is sufficient to effect treatment, as defined below, for cancer, inflammation, or renal disease in the mammal. The amount of a compound of formula (I) which constitutes a “therapeutically effective amount” will vary depending on the compound, the condition and its severity, and the age of the mammal to be treated, but can be determined routinely by one of ordinary skill in the art having regard to his own knowledge and to this disclosure.

“Treating” or “treatment” as used herein covers the treatment of a hyperproliferative disease as disclosed herein, in a mammal, preferably a human, and includes:

(i) preventing cancer, inflammation, or renal disease from occurring in a mammal, in particular, when such mammal is predisposed to the condition but has not yet been diagnosed as having it;

(ii) inhibiting cancer, inflammation, or renal disease, i.e., arresting its development; or

(iii) relieving cancer, inflammation, or renal disease, i.e., causing regression of the condition.

The compounds of formula (I), or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)- isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The nomenclature used herein for the compounds of formula (I) is a modified form of the I.U.P.A.C. nomenclature system wherein the compounds are named herein as derivatives of the isothiazole moiety.

### METHODS OF USE

This invention is directed to methods of using compounds of formula (I), as set forth above in the Summary of the Invention, and pharmaceutical compositions containing compounds of formula (I) in treating hyperproliferative conditions. Thus, the methods disclosed herein are useful in treating disorders and physiological conditions associated with hyperproliferation and tissue remodelling or repair when administered to a subject in need of such treatment. Of particular interest are hyperproliferative disorders associated with cellular modulation of protein phosphorylation states, i.e. altered activity of phosphorylation modifying enzyme(s), e.g. protein tyrosine kinases and protein tyrosine phosphatases.

In one aspect of the invention, compounds and pharmaceutical compositions of the invention are used to inhibit the activity of PTPN12. This enzyme has been associated with alterations in the phosphorylation state of cellular proteins.

The compounds and pharmaceutical compositions of the invention are administered to a subject having a cancer or a pathological inflammation in order to inhibit tumor growth by impeding cell division, and to decrease inflammation by inhibiting cell adhesion and cell migration. In addition, the methods of the invention may be used in association with restoring the normal foot process architecture of podocytes in glomerular diseases associated with proteinuria (Reiser, J. et al., Rapid Communication, Kidney int. (2000), Vol. 57, No. 5, pp. 2035-2042).

The methods of the invention can be used prophylactically (i.e., to prevent the disorder of interest from occurring) or therapeutically (i.e., to inhibit or relieve the disorder). As used herein, the term “treating” is used to refer to both prevention of disease, and treatment of preexisting conditions. The prevention of symptoms is accomplished by administration of the compounds and pharmaceutical compositions of the invention prior to development of overt disease, e.g., to prevent the regrowth of tumors, prevent metastatic growth, diminish restenosis associated with cardiovascular surgery, to prevent or reduce cell migration leading to inflammation and associated tissue damage. Alternatively, the compounds and pharmaceutical compositions of the invention may be administered to a subject in need thereof to treat an ongoing disease, by stabilizing or improving the clinical symptoms of the patient.

The subject, or patient, may be from any mammalian species, e.g. primates, particularly humans; rodents, including mice, rats and hamsters; rabbits; equines; bovines; canines; felines; etc. Animal models are of interest for experimental investigations, providing a model for treatment of human disease.

Hyperproliferative disorders refers to excess cell proliferation, relative to that occurring with the same type of cell in the general population and/or the same type of cell obtained from a patient at an earlier time. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Hyperproliferative cell disorders include cancers; blood vessel proliferative disorders such as restenosis, atherosclerosis, in-stent stenosis, vascular graft restenosis, etc.; fibrotic disorders; psoriasis; inflammatory disorders, e.g. arthritis, etc.; glomerular nephritis; endometriosis; macular degenerative disorders; benign growth disorders such as prostate enlargement and lipomas; and autoimmune disorders. Cancers of particular interest include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; sarcomas, melanomas, adenomas; benign lesions such as papillomas, and the like.

Other hyperproliferative disorders that may be associated with altered activity of phosphorylation modifying enzyme(s) include a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

Disorders and conditions where there is hyperproliferation and/or tissue remodelling or repair of reproductive tissue, e.g. uterine, testicular and ovarian carcinomas, endometriosis, squamous and glandular epithelial carcinomas of the cervix, etc. are reduced in cell number by administration of the compounds and pharmaceutical compositions of the invention. Other disorders and conditions of interest relate to epidermal hyperproliferation, tissue remodelling and repair. For example, the chronic skin inflammation of psoriasis is associated with hyperplastic epidermal keratinocytes.

Other disorders of interest include inflammatory disorders and autoimmune conditions including, but not limited to, psoriasis, rheumatoid arthritis, multiple sclerosis, scleroderma, systemic lupus erythematosus, Sjogren's syndrome, atopic dermatitis, asthma, and allergy. Target cells susceptible to the treatment include cells involved in instigating autoimmune reactions as well as those suffering or responding from the effects of autoimmune attack or inflammatory events, and include lymphocytes and fibroblasts.

The susceptibility of a particular cell to treatment according to the invention may be determined by in vitro testing. Typically, a culture of the cell is combined with a subject compound at varying concentrations for a period of time sufficient to allow the active agents to induce cell death or inhibit migration, usually between about one hour and one week. For in vitro testing, cultured cells from a biopsy sample may be used.

The dose will vary depending on mode of administration, specific disorder, patient status, etc. Typically a therapeutic dose will be sufficient to substantially decrease the undesirable cell population in the targeted tissue, while maintaining patient viability. Treatment will generally be continued until there is a substantial reduction, e.g. at least about 50%, decrease in the clinical manifestation of disease, and may be continued until there are essentially none of the undesirable cellular activity detected in the relevant tissue.

The compounds of formula (I) may also find use in the specific inhibition of signaling pathways mediated by protein tyrosine phosphatases, for example, PTPN12, and as a “positive” control in high throughput screening for other modulating compounds. In particular, this invention directed to methods of using compounds of formula (I) and pharmaceutical compositions containing such compounds in treating cancer or inflammation associated with PTPN12 activity.

PTPN12 contains a proline rich motif at its C-terminal and can bind to p130^{cas}, which is a focal adhesion associated protein containing an SH3 domain. In normal cells, p130^{cas} becomes highly phosphorylated following integrin dependent activation of the fak and src kinases. This phosphorylation appears to allow a series of tyrosine dependent signalling that has among other consequences the actin filament reorganization. Because of the importance of integrin signalling in the cell cytoskeleton, motility and transformation, the action of PTPN12 on p130^{cas} may have dramatic consequences in mammalian development as well as in some physiopathological events. The process of cell migration is crucial for the correct development of a mammalian embryo. In an adult organism, cell migration plays an important role in events like invasion of a wounded space by fibroblasts and endothelial cells and translocation of lymphocytes and neutrophiles to an inflammation site. In cancer, tumor cells also have to migrate in order to reach the circulatory system and disperse throughout the organism. Takekawa, M. et al., FEBS Lett.(1994), Vol. 339, pp.222-228 discloses aberrant transcripts of PTPN12 in cancer cells. The effect of PTPN12 levels on fibroblast motility is described in Garton et al. (1999) J. Biol. Chem. 274(6):3811-3818. Davidson et al. (2001) EMBO. J. 20(13):3414-26 discusses a connection of PTPN12 with inflammation. The relationship between PTPN12 and podocyte regulation in kidney is described in Reiser, J. et al., Rapid Communication, Kidney Int. (2000), Vol. 57, No. 5, pp. 2035-2042.

PTPN12 is involved in signalling pathways for such important cellular activities as responses to extracellular signals and cell cycle checkpoints. Inhibition of PTPN12 provides a means (for example, by blocking the effect of an extracellular signal) of intervening in these signalling pathways, which are associated with a variety of pathological or clinical conditions. PTPN12 is associated with cell adhesion, cell division and cell migration and thus is implicated in cancer and inflammation.

The compounds of formula (I) may also find use as affinity reagents for the isolation and/or purification of phosphatases using the biochemical affinity of the enzyme for inhibitors that act on it. The compounds are coupled to a matrix or gel. The coupled support is then used to separate the enzyme, which binds to the compound, from a sample mixture, e.g., a cell lysate, which may be optionally partially purified. The sample mixture is contacted with the compound coupled support under conditions that minimize non-specific binding. Methods known in the art include columns, gels, capillaries, etc. The unbound proteins are washed free of the resin and the bound proteins are then eluted in a suitable buffer.

The compounds of formula (I) may also be useful as reagents for studying signal transduction or any of the clinical disorders listed throughout this application, and for use as a positive control in high throughput screening.

### ADMINISTRATION OF THE COMPOUNDS AND PHARMACEUTICAL COMPOSITIONS OF THE INVENTION

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disorder or condition associated with hyperproliferation and tissue remodelling or repair in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, e.g., inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel™, corn starch and the like; lubricants such as magnesium stearate or Sterotex™; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or a fatty oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 80% of the compound of the invention. Preferred pharmaceutical compositions and preparations according to the present invention are prepared so that a parenteral dosage unit contains between 0.01 to 1% by weight of the compound of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation, may determine preferred aerosols.

Whether in solid, liquid or gaseous form, the pharmaceutical composition of the present invention may contain one or more known pharmacological agents used in the treatment of cancer or inflammation in a mammal, particularly, cancer or inflammation associated with hyperproliferation and tissue remodelling or repair.

The pharmaceutical compositions of the invention may be prepared by methodology well known in the pharmaceutical art. For example, a pharmaceutical composition intended to be administered by injection can be prepared by combining a compound of the invention with water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is from about 0.1 mg to about 20 mg/kg of body weight per day of a compound of the invention, or a pharmaceutically acceptable salt thereof; preferably, from about 0.1 mg to about 10 mg/kg of body weight per day; and most preferably, from about 0.1 mg to about 7.5 mg/kg of body weight per day.

### PREFERRED EMBODIMENTS OF THE INVENTION

Of the various methods of treating cancer or inflammation in a mammal as set forth above in the Summary of the Invention, a preferred method is that method wherein the cancer or inflammation is associated with hyperproliferation or tissue remodelling or repair. Another preferred method is that method wherein the cancer or inflammation is associated with the activity of an enzyme selected from the group consisting of PTPN12.

Of the various methods of treating a mammalian cell with a compound of formula (I) as set forth above in the Summary of the Invention wherein the method comprises administering the compound of formula (I) to a mammalian cell and the compound of formula (I) is capable of inhibiting the activity of PTPN12 within the mammalian cell, a preferred method is that method wherein the mammalian cell is treated in vitro. Another preferred method is that method wherein the mammalian cell is treated in vivo. Another preferred method is that method wherein the inhibition of activity results in a reduction of cell adhesion. Another preferred method is that method wherein the inhibition of activity results in a reduction of cell division. Another preferred method is that method wherein the inhibition of activity results in a reduction of cell migration. Another preferred method is that method wherein the inhibition of activity results in control of tumor growth. Another preferred method is that method wherein the inhibition of activity results in control of lymphocyte activation.

Of the various methods of treating a mammal as set forth above in the Summary of the Invention, a preferred method is that method wherein the mammal is a human.

Of the various methods or pharmaceutical compositions set forth herein and above in the Summary of the Invention, a preferred method or pharmaceutical composition is wherein R¹ is alkyl or alkenyl.

Another preferred method or pharmaceutical composition is that method or pharmaceutical composition wherein the R¹ substituent of the compound of formula (I) or formula (II) is aryl, aralkyl or aralkenyl.

Another preferred method or pharmaceutical composition is that method or pharmaceutical composition wherein the R¹ substituent of the compound of formula (I) or formula (II) is cycloalkyl, cycloalkylalkyl or cycloalkylalkenyl.

Another preferred method or pharmaceutical composition is that method or pharmaceutical composition wherein the R¹ substituent of the compound of formula (I) or formula (II) is haloalkyl, haloalkenyl, haloalkoxyalkyl or haloalkoxyalkenyl.

Another preferred method or pharmaceutical composition is that method or pharmaceutical composition wherein the R¹ substituent of the compound of formula (I) or formula (II) is —R⁴—N═N—O—R⁵.

Another preferred method or pharmaceutical composition is that method or pharmaceutical composition wherein the R¹ substituent of the compound of formula (I) or formula (II) is —N(R⁶)₂.

Another preferred method or pharmaceutical composition is that method or pharmaceutical composition wherein the R¹ substituent of the compound of formula (I) or formula (II) is heterocyclylalkyl.

Of the various methods or pharmaceutical compositions set forth herein and above in the Summary of the Invention, a preferred method or pharmaceutical composition is wherein the R² substituent of the compound of formula (I) or formula (II) is hydrogen, alkyl or alkenyl.

Another preferred method or pharmaceutical composition is that method or pharmaceutical composition wherein the R² substituent of the compound of formula (I) or formula (II) is aryl, aralkyl or aralkenyl.

Another preferred method or pharmaceutical composition is that method or pharmaceutical composition wherein the R² substituent of the compound of formula (I) or formula (II) is cycloalkyl, cycloalkylalkyl or cycloalkylalkenyl.

Another preferred method or pharmaceutical composition is that method or composition wherein the R² substituent of the compound of formula (I) or formula (II) is halo, haloalkyl or haloalkenyl.

Another preferred method or pharmaceutical composition is that method or composition wherein the R² substituent of the compound of formula (I) is cyano, nitro or —R⁴—N═N—O—R⁵ or wherein the R² substituent of the compound of formula (II) is nitro or —R⁴—N═N—O—R⁵.

Another preferred method or pharmaceutical composition is that method or composition wherein the R² substituent of the compound of formula (I) or formula (II) is —OR⁶.

Another preferred method or pharmaceutical composition is that method or composition wherein the R² substituent of the compound of formula (I) or formula (II) is —C(O)OR⁶.

Another preferred method or pharmaceutical composition is that method or composition wherein the R² substituent of the compound of formula (I) or formula (II) is —N(R⁶)₂.

Another preferred method or pharmaceutical composition is that method or composition wherein the R² substituent of the compound of formula (I) or formula (II) is —C(O)N(R⁶)₂ or —N(R⁶)C(O)OR⁵.

Another preferred method or pharmaceutical composition is that method or composition wherein the R² substituent of the compound of formula (I) or formula (II) is heterocyclyl or heterocyclylalkyl.

Of the various methods or pharmaceutical compositions set forth herein and above in the Summary of the Invention, a preferred method or pharmaceutical composition is that method or composition wherein t in the compound of formula (I) or formula (II) is 0.

Another preferred method or pharmaceutical composition is that method or composition wherein t in the compound of formula (I) or formula (II) is 1.

Another preferred method or pharmaceutical composition is that method or composition wherein t in the compound of formula (I) or formula (II) is 2.

Preferred compounds of formula (II) as set forth above in the Summary of the Invention are those compounds of formula (II) in the preferred methods and pharmaceutical compositions set forth above.

### PREPARATION OF THE COMPOUNDS OF FORMULA (I)

Compounds of formula (I) in the methods and pharmaceutical compositions of the invention may be prepared according to methods known to one skilled in the art, or by the methods similar to those disclosed in published U.S. Pat. Nos. 5,578,622; 5,952,359; 5,438,053; 4,094,880 (all of which are incorporated in full by reference herein), or by methods similar to the method described below.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein.

The use of protecting groups is described in detail in Green, T. W. and P. G. M. Wutz, Protective Groups in Organic Synthesis (1991), 2nd Ed., Wiley Interscience. The protecting group may also be a polymer resin such as a Wang resin or a 2-chlorotrityl chloride resin.

It will also be appreciated by those skilled in the art, although such protected derivatives of compounds of formulae (I), as described above in the Summary of the Invention, may not possess pharmacological activity as such, they may be administered to a mammal with cancer or inflammation and thereafter metabolized in the body to form compounds of the invention which are pharmacologically active. Such derivatives may therefore be described as “prodrugs”. All prodrugs of compounds of formula (I) are included within the scope of the invention.

In the following Reaction Scheme, R¹, R² and R³ are as described in the Summary of the Invention for compounds of formula (I), and each X and each X′ are independently halo. It is understood, however, that one of ordinary skill in the art would be able to prepare other compounds of formula (I) and formula (II) from methods known to one skilled in the art.

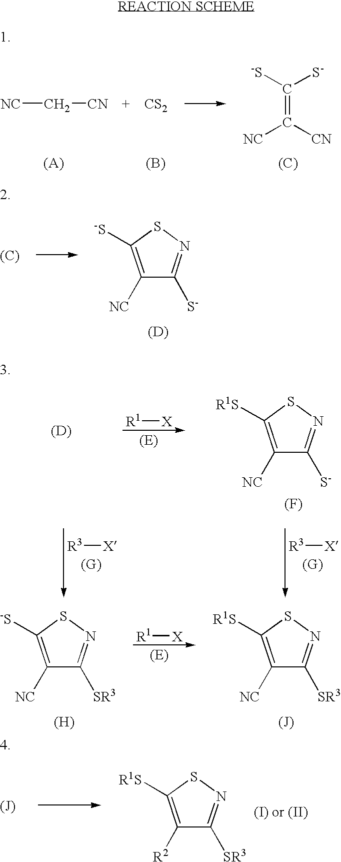

In this general scheme, starting components may be obtained from sources such as Aldrich, or synthesized according to sources known to those of ordinary skill in the art, (see, e.g., Smith and March, March's Advanced Organic Chemistry: Reactions, Mechanisms, and Structure, 5th edition (Wiley Interscience, New York). Groups R¹ through R³ are selected from components as indicated in the specification heretofore.

A dimercaptomethylenemalononitrile of formula (C) can be prepared under standard coupling conditions according to schemes known to those of ordinary skill in the art (see, e.g., Hatchard, J. Org. Chem., vol. 29, pp. 665-668 (1964)). As one example, a compound of formula (C) is formed according to Step 1 of the reaction scheme depicted herein, whereby 1 molar equivalent of the malononitrile of formula (A) is slowly combined with about 2 molar equivalents of sodium hydroxide in an alcohol such as ethyl alcohol, with stirring, at about 10° C. to 15° C. About 2 molar equivalents of the carbon disulfide of formula (B) is then added dropwise to the admixture, with cooling, for about 30 minutes, followed by additional stirring of the admixture for about 1 hour at ambient temperature. The resulting reaction product is filtered, washed with a solvent such as ethyl alcohol, and dried, to afford the disodium salt of formula (C).

In another example, a compound of formula (C) is formed according to Step 1 of the reaction scheme depicted herein, whereby a mixture composed of 1 molar equivalent of the malononitrile of formula (A) and about 1 molar equivalent of the carbon disulfide of formula (B) in acetonitrile is combined with about 2 molar equivalents of triethylamine over about 15 minutes. After stirring the admixture for about 30 minutes, and diluting the admixture with a solvent such as ethyl ether, the resulting reaction product is filtered, washed with a solvent such as ethyl ether, and dried, to afford the bis(triethylammonium) salt of formula (C).

A dimercaptoisothiazolecarbonitrile compound of formula (D) may be prepared under standard cyclization conditions according to schemes known to those of ordinary skill in the art (see, e.g., Hatchard, J. Org. Chem., vol. 29, pp. 665-668 (1964)). For instance, a compound of formula (D) is formed according to Step 2 of the reaction scheme depicted herein, whereby a mixture consisting of a compound of formula (C) (at a concentration of about 0.2 moles/liter) and sulfur (at a concentration of about 0.2 moles/liter) in methanol is heated under reflux for about 15 minutes. After heating, the reaction product is filtered, evaporated to dryness, and further dried in a vacuum oven over diphosphorus pentoxide, to afford a dimercaptoisothiazolecarbonitrile divalent salt of formula (D).

An S-substituted mercaptoisothiazolecarbonitrile of formula (J) may be prepared under standard addition conditions according to schemes known to those of ordinary skill in the art (see, e.g., Hatchard, J. Org. Chem., vol. 29, pp. 665-668 (1964)), wherein R¹ and R³ are selected from components as indicated in the specification heretofore. For example, a compound of formula (J) wherein R¹ and R³ are alkyl may be formed according to Step 3 of the reaction scheme depicted herein, whereby a reaction vessel is charged with 1 molar equivalent of a compound of formula (D) and about 1 molar equivalent of an alkyl halide of formula (E) in a suitable solvent (such as methanol). In optional aspects the admixture is combined in a dropwise fashion over the course of about 15 to 30 minutes; the admixture is heated, optionally under reflux, for about 15 to 60 minutes; and/or the admixture is stirred for about 30 to 60 minutes, preferably at ambient temperature.

The reaction vessel is then further charged with about 1 molar equivalent of an independently-selected alkyl halide of formula (G), optionally in a solvent such as methanol. In optional aspects the compound of formula (G) is charged in a dropwise fashion over the course of about 15 to 30 minutes; the admixture is heated, optionally under reflux, for about 15 to 60 minutes; the admixture is stirred for about 30 to 60 minutes, preferably at ambient temperature; and/or the admixture is diluted with a solvent selected so as to promote precipitation of the reaction product. In an optional aspect, the compound of formula (D) may first undergo addition via treatment with the compound of formula (G), followed by addition via treatment with the compound of formula (E), under the reaction conditions indicated. In a separate optional aspect, the compounds of formulae (E) and (G) may be reacted simultaneously with the compound of formula (D), under the reaction conditions indicated. Other compounds of formulae (E) and (G) wherein R¹ and R³ are selected from components as indicated in the specification heretofore may be reacted in a manner similar to that described herein.

The resulting reaction product is filtered and dried via solvent evaporation. In optional aspects, the reaction product is then recrystallized and isolated; and/or the reaction product is then washed with a solvent wherein the product is relatively insoluble, before being allowed to dry. The resulting product is a compound of formula (J), although typically the product is a mixture of compounds consisting of a compound of formula (J) plus additional compound(s) of formula J wherein R¹ is replaced by R³, and/or R³ is replaced by R¹. Components of such product mixtures may then be separated from each other and purified through the use of a preparative separation and isolation technique such as high performance liquid chromatography (HPLC).

A substituted mercaptoisothiazolecarbonitrile of formula (I) may be prepared under standard addition conditions according to schemes known to those of ordinary skill in the art (see, e.g., Hatchard, J. Org. Chem., vol. 29, pp. 665-668 (1964)), wherein R¹, R², and R³ are selected from components as indicated in the specification heretofore. For example, a compound of formula (I) wherein R² is —C(O)—N(R⁶)₂ where each R⁶ is hydrogen may be formed according to Step 4 of the reaction scheme depicted herein, whereby a solution of a compound of formula (J) in concentrated sulfuric acid is heated to about 60° C. to 70° C. for about 4 hours, then poured into ice water. The resulting reaction product is filtered and allowed to dry, affording an isothiazolecarboxamide compound of formula (I).

In another example, a compound of formula (I) wherein R² is —C(O)OR⁶ where R⁶ is hydrogen may be formed by combining 1 molar equivalent of an isothiazolecarboxamide of formula (I), water, concentrated sulfuric acid, and about 1.5 molar equivalents of aqueous sodium nitrite at about 5° C. to 10° C. for about 15 minutes. After using an apparatus such as a steam bath to warm the reaction mixture for about 30 minutes, the mixture is poured into ice water to produce the crude reaction product. The crude product is filtered and washed with water. Optionally, the crude product is further redissolved in aqueous alkaline solution such as sodium carbonate, filtered, acidified by the addition of a dilute acidic solution such as aqueous hydrochloric acid, and extracted with an organic solvent such as methylene chloride. The solvent of the reaction product is then allowed to evaporate to dryness, affording an isothiazolecarboxylic acid of formula (I).

In still another example, a compound of formula (I) wherein R² is —C(O)OR⁶ where R⁶ is as set forth above in the Summary of the Invention except that R⁶ is not be hydrogen may be formed by combining an isothiazolecarbonyl chloride of formula (I) and hot alkyl alcohol. After sufficient time so as to allow reaction to occur, the solvent of the reaction mixture is then evaporated to dryness, affording an isothiazolecarboxylate of formula (I).

In yet another example, a compound of formula (I) wherein R² is —C(O)—N(R⁶)₂ where each R⁶ is alkyl may be formed by combining an isothiazolecarbonyl chloride of formula (I), an alkylamine, and a solvent such as ethyl ether. After sufficient time so as to allow reaction to occur, the solvent of the reaction mixture is then evaporated to dryness, affording an N-alkylisothiazolecarboxamide of formula (I).

In yet another example, a compound of formula (I) wherein R² is —C(O)—N(R⁶)₂ where each R⁶ is aralkyl may be formed by combining 1 molar equivalent of an isothiazolecarbonyl chloride of formula (I), about 6 molar equivalents of the appropriate aniline, and a solvent such as ethyl ether. The reaction mixture is heated under reflux for about 15 minutes, then diluted with water and an organic solvent such as methylene chloride. After extraction of the reaction product into the organic phase of the solvent system, the organic phase is washed with an acidic solution such as aqueous hydrochloric acid. Subsequent evaporation of the organic phase and optional recrystallization in an organic solvent such as methanol affords an N-phenylisothiazolecarboxamide of formula (I).

Other compounds of formula (I) wherein R² is selected from components as indicated in the specification heretofore may be produced in a manner similar to those described herein.

Corresponding compounds of formula (I) and formula (II) as set forth above in the Summary of the Invention where t is 1 or 2 may be prepared by reacting a corresponding compound of formula (I) as set forth above or a corresponding compound of formula (II) with the appropriate amount of an oxidizing agent, or by methods known to one skilled in the art.

### EXAMPLES

### Example 1

### Enzyme Preparation and Use

A. PTPN12

PTPN12 was cloned in the IMPACT™ (New England BioLabs) bacterial expression system. The IMPACT™ Protein Purification System was purchased commercially from New England BioLabs.

1. Cloning of truncated Human PTPN12 into pTWIN-II expression

vector

Expression of human truncated PTPN12 (PTP-PEST-N) as a fusion protein required that the cDNA be ligated into the polyclonal site situated in frame and upstream of the intein gene of the IMPACT™ expression vector pTWIN-II. The truncated version was used as it was far easier to handle and gave parallel results to the full length protein in comparison testing. For the purpose of simplicity, PTP-PEST-N will be used interchangeably with PTPN12 in these Examples.

The PTPN12 coding sequence was generated by polymerase chain reaction (PCR) using gene-specific primers.

2. Human PTPN12 Expression and purification

Active PTPN12 enzyme is expressed from the IMPACT™ vector system in the bacterial strain ER2566. Recombinant PTPN12 protein is purified from bacterial cells using affinity chromatography on chitin-agarose beads followed by a chemical process whereby PTPN12 is released from its affinity tag. A complete quantitative and qualitative analysis of the protein is monitored using Coomassie blue staining of SDS-PAGE separated preparations and by PTPN12-specific Western blotting. PTPN12 is produced at levels in the range of 0.1-0.5 mg per litre of bacterial cell culture.

3. PTPN12 In Vitro Phosphatase Assay

Biochemical analysis is performed on recombinant human PTPN12 fusion protein. Typically, the PTPN12 preparations are found to exhibit protein phosphatase activity in the order of 1500 to 2500 pmol/min/μg measured as phosphate release from a synthetic tyrosine phosphorylated peptide. This activity is considered to be in the high range as compared to other recombinant protein tyrosine phosphatases. PTPN12 preparations were subsequently used extensively in in vitro assays for the initial discovery of compounds having the ability to inhibit PTPN12 activity.

### Example 2

### In Vitro Activity Profile for Phosphatases

Compounds were tested in the following assay for their ability to inhibit the activity of the desired phosphatase.

A. Reagent Preparation:

1. Malachite Green-Ammonium Molybdate Reagent

Two solutions were first prepared. Solution 1 contained 4.2 % ammonium molybdate tetrahydrate (Sigma, Cat# A-7302) in 4 N HCI. Solution 2 contained 0.045% Malachite green (Sigma, Cat. # M-9636). The two solutions were mixed as follows: 250 mL of solution 1 and 750 mL solution 2 with constant stirring for 20 min. The resulting mixture was filtered through 0.22 μM filter (one can use Nalgene™ bottle top vacuum filters Cat # 28199-317). The solution was stored in a brown bottle at 4° C.

B. Preparation of 1 mM ppC SRC 60 Substrate

The peptide sequence: TSTEPQY(PO₄)QPGENL was prepared by conventional methods. Of this,154 mg was dissolved in 100 mL dH₂O and the solution vortexed until the peptide dissolved completely. The ppC SRC 60 was then stored in 1 mL aliquots at −20° C. This is the “Substrate” used for preparing the substrate working stock solution.

C. Procedure for Assay

The enzyme (phosphatase) activity was determined in a reaction that measured phosphate relase from tyrosine phospho-specific peptides using a method first described by Harder et al., Biochem. J. (1994), Vol. 298, pp. 395-401. This is a non-radioactive method for measuring free phosphate by the malachite green method first described by Van Veldhoven and Mannaerts, Anal Biochem. (1987), Vol.161, pp. 45-48. 10× assay buffer (250 mM Tris:100 mM, β-Mercaptoethanol, 50 mM EDTA; pH 7.2) was diluted to 5× concentration with distilled H₂O (dH₂O). Then 71.4 μM of substrate working stock solution was prepared in dH₂O.

In a microcentrifuge tube, the required volume of enzyme stock was pipetted, diluted with the required volume of 5× assay buffer and mixed.

The colour reagent was prepared by thoroughly mixing 10 mL Malachite Green-ammonium molybdate reagent and 100 μL of 1% Tween-20 (One mL Tween-20 (BDH, #06435) dissolved in 99 mL dH₂O) into a reagent reservoir and storing at room temperature. Approximately 10 mL of colour reagent is required per assay plate, or 100 μL per well.

Sample Compound Preparation

In a Falcon 96 well plate the sample compound was diluted in 1% DMSO (One mL DMSO (Sigma, Cat. # D-8779) dissolved in 99 mL dH₂O and stored at room temperature) such that the concentration of the sample compound working stock solution is ten times the final desired concentration of the compound in the assay.

The working stock solution was prepared as per the required concentration of sample compound in the assay.

The negative control consisted of 5 μl 1% DMSO and 35 μL substrate working stock solution and 10 μL diluted enzyme, per well), and was placed in the first column of wells on the plate. The last column of wells on the plate was reserved for an enzyme blank, which consisted of 5 μL 1% DMSO, 35 μL substrate working stock solution, and 10 μL 5× assay buffer, per well. Test samples were placed in columns 2-11 and consisted of 5 μL sample in 1% DMSO, 35 μL substrate working stock solution, and 10 μL of diluted enzyme, per well, at the desired concentration. Using the repeater function of a Biohit™ multichannel pipettor, 5 μL of 100 μM sample from the Falcon plate columns was added to corresponding Costar™ assay plate columns.

Then 5 μL 1% DMSO was added to column 1 & 12, and 10 μL of 5× assay buffer to column 12.

Using a multichannel pipettor, 35 μL of 71.4 μM ppC-SRC 60 substrate was added to all assay wells, then 10 μL of appropriately diluted enzyme was added to the wells on a column by column basis, pausing 5 seconds between columns. Timing started at the first addition.

The assay plate was incubated at room temperature (21° C.) for 15 minutes. The reaction was “stopped” by adding 100 μL colour reagent on a column by column basis, pausing 5 seconds between columns. Colour was allowed to develop for at least 15 minutes, but no longer than two hours, at room temperature. The plate was “read” on Bio-tek Instruments EL312e™ microplate Bio-Kinetics™ reader at 590 nm and the data collected as per instrument manual.

Data analysis was performed as follows. The blank and negative controls were read, and blanks were subtracted from the average of negative control values and sample values, and the % inhibition was expressed by the following formula:
% Inhibition=100−[corrected sample reading/corrected Negative Control reading*100].

TABLE 1
In Vitro Assay Results at 20 μm Concentration

| | % | |
|---|---|---|
| | Inhibition of | |
| Compound Name | PTPN12 | IC₅₀ |
| (3,5-Bis-methylsulfanyl-isothiazol-4-yl)- | 51 | Not calculated |
| piperidin-1-yl-methanone | | |
| 5-Benzylsulfanyl-3-methylsulfanyl- | 94 | 1.5 |
| isothiazole-4-carboxylic | | |
| acid amide | | |
| 2-{4-Cyano-3-[(4-sulfamoyl- | 73 | Not calculated |
| phenylcarbamoyl)-methylsulfanyl]- | | |
| isothiazol-5-ylsulfanyl}-N-(4- | | |
| sulfamoyl-phenyl)-acetamide | | |
| 3,5-Bis-(2-ethanesulfonyl-ethylsulfanyl)- | 60 | Not calculated |
| isothiazole-4-carbonitrile | | |
| 1-(3,5-Bis-methanesulfonyl-isothiazole-4- | 63 | Not calculated |
| carbonyl)-3-(2-chloro-phenyl)-urea | | |

### Example 3

### Caspase-3 Assay

This is a selectivity assay, which provides information about the specificity of the compounds. A cysteine residue located within the active pocket of the catalytic domain that is conserved between the serine proteases like caspase-3 and the protein tyrosine phosphatases like PTPN12 has a thiol group within this cysteine amino acid that may interact with specific R-groups with inhibiting compounds with in vitro enzyme inhibiting activity. Also, caspase inhibition can prevent apoptosis from occurring in some cases via the caspase cascade. As the potential for cross-inhibitory activity and nonselectivity exists, the caspase-3 assay is performed on PTN12 inhibitors.

The Calbiochem caspase-3 assay kit was utilized according to package insert instructions. The assay is useful for screening for caspase-3 inhibitors measuring the protease activity of caspase-3 and other caspase-3-like activities. Cleavage is monitored calorimetrically by measuring the increase in absorbance at 405 nm. Assays were performed in a 96-well microtiter plate format.

TABLE 2
Inhibition of PTPN12 v. Caspase-3

| | % Inhibition of | % Inhibition of |
|---|---|---|
| Compound Name | PTPN12 | Caspase 3 |
| 5-Benzylsulfanyl-3-methylsulfanyl- | 94 at 20 μM | 4 at 10 μm |
| isothiazole-4-carboxylic | | 14 at 100 μm |
| acid amide | | |

### Example 4

### Cell Migration In A Boyden Chamber

A range of cell lines are used in this assay, particularly the prostate cancer cell line PC3 and PTPN12 mouse embryonic fibroblasts (MEFs). The role of PTPN12 in migration was established based on the observations of PTPN12 negative MEFs. Cell adhesion and migration are dynamic biological activities involving the assembly and disassembly of a large number of extracellular and intracellular molecules, for example, actin, which are regulated in turn by protein phosphorylation. Hence locking the system in a phosphorylated (inhibition of phosphatases) or dephosphorylated (inhibition of kinases) state has a profound effect on the assembly/disassembly process and ultimately migration. Migration is reduced in PTPN12 knock-out MEFs. By extension, a PTPN12 inhibitor should reduce cell migration in a Boyden chamber. Therefore, as a readout for PTPNI2 activity, the following assay is designed to analyze cell migration in Boyden chambers. The Boyden assay is an experiment used to determine the capacity of a cell type to migrate on extracellular matrix. Unless otherwise indicated, all procedures are performed under sterile conditions in a flow laminar hood and all incubations at 37° C. are performed in the CO₂ incubator.

A. Reagents

1. Staining Solution.

Calcein AM (Molecular Probes, Cat# C-1430) stain is prepared at 0.5 ug/ml in Hanks Buffered Saline solution (GIBCO/BRL, Cat#14170-112).

2. Fibronectin Solution

A stock solution of fibronectin is prepared by dissolving 5 mg of fibronectin: (Sigma, Cat: F-2006) in 5 mL of sterile phosphate-buffered solution (PBS) by up and down agitation with a P1000™ pipette. The working solution is prepared by mixing 100 μl of this stock solution with 10 mL of sterile PBS.

B. Assay (Tumour cell lines)

For tumour cell lines, Stock cells (i.e. PC3 cells) are grown to 50-70% confluency in T175 flasks. Cells are trypsinized and a suspension prepared to a concentration of 2×10⁵/ml in media without serum. To the top chamber of each well of the HTS FluoroBlok™ 24-well insert system plates (Cat# 351158) is added 450 μl of cell suspension (or media for controls). Compounds for testing are prepared as 10× stocks in serum-free media from DMSO stocks, with a maximum final DMSO concentration of 0.25%. 50 μl of compound (or DMSO control) is then added to each top chamber, while 750 μl of media containing 10% fetal bovine serum is added to the bottom chamber as the chemoattractant. The plates are incubated for 20-24 hours at 37° C., 5% CO₂. Following incubation, the insert plate is transferred into a second 24-well companion plate containing 0.5 ml of 5 ug/ml calcein AM in HBSS and incubated for 1 hour at 37° C., 5% CO₂. Fluorescence of migrated cells is read in a Fluoroskan™ Ascent FL™ reader (or equivalent) with bottom reading at excitation/emission wavelength of 485/538 nm. Only those cells that have migrated through the pores of the FluorBlok™ membrane will be read. For MEFs, the plates are coated on both sides of the membrane with 10 mg/mL fibronectin solution for 18 hours at 4° C. After incubation, the coating solution is removed by aspiration and the excess is washed twice with PBS. Cell seeding and detection are then performed as described for tumour cell lines.

C. Data Analysis

Data is expressed as fluorescence unit (FU) from the sum of middle 25 areas per 24-well or as percentage of migration inhibition by following formula: % of invasion inhibition =100 - FU of compound treated cell invasion/FU of DMSO treated cell invasion times 100. Background is subtracted from all values, with background being represented by the media only controls.

### Example 5

### The Status of p130^{cas} Phosphorylation on Western Blots

Phosphotyrosine profiling of PTPN12-heterozygote and PTPN12-knockout mouse fibroblasts showed that a protein migrating at 130 kDa is constitutively hyperphosphorylated in the knockout cells (Côté, J. F., et al., Biochemistry (1998), Vol. 37, No. 38, pp. 13128-13137). This protein was identified as being p130^{cas}, a protein found in focal adhesion complexes. It also appeared that the hyperphosphorylation of p130^{cas} in the PTPN12 knockout cells resulted in defective cell motility and focal adhesion turnover (Angers-Loustau et al., 1999).

This following assay measures p130^{cas} phosphorylation status as a readout of PTPN12 activity. Briefly, the general tyrosine phosphorylation state of all cellular proteins is reduced by incubating the cells in suspension and then plating the cells onto fibronectin-coated plates, thereby stimulating tyrosine phosphorylation through the integrin pathway. Following cell lysis, p130^{cas} immunoprecipitation and Western blotting using 4G10™ antiphosphotyrosine antibody are used to measure the tyrosine phosphorylation status of p130^{cas}. A low level of p130^{cas} tyrosine phosphorylation is indicative of a high PTPN12 activity. The assay is performed using PTPN12 knockout and heterozygote mouse fibroblasts.

A. Materials

1. PTPN12 +/−mouse fibroblasts (AC4 +/−) and PTPN12 −/− mouse fibroblasts (AC6 −/−) as kindly provided by Michel Tremblay and colleagues from the McGill Cancer Centre at McGill University.

2. RIPA Buffer is made by mixing 50 mM Tris-HCl pH 7.2, 150 mM NaCl, 0.1% SDS (BioShop, Cat#: SDS 001), 0.5% Sodium deoxycholate 10% solution (Sigma, Cat: D-6750), 1% NP-40 (BDH Laboratory Supplies, Cat: 56009 2L), 1 mM sodium vanadate (Fisher Scientific, Cat: S454-50) 200 mM solution, and “complete protease inhibitor mixture” from Roche (Cat. 1836153).

3. SDS Sample Buffer is prepared by mixing 62.5 mM Tris-HCl pH 6.8, 20% glycerol (BioShop, Cat#: Gly 001), 2 % SDS, 5% β-mercaptoethenol (Acros Organics, Cat#: 12547-2500), and 0.025% bromophenol blue (EM Science, OmniPur™).

B. Fibronectin Stimulation

6-Well plates (Fisher Scientific, Cat: 08-772-1B, Falcon No. 3530) are coated for 18 hours at 4° C. with a 10 mg/mL fibronectin solution (Sigma, Cat: F-2006, Lot: 109H7602) (density of 1 g/cm²). A volume of 950 μl of the fibronectin solution is added to each well. The plates are washed 2 times by adding 2 mL of PBS at ambient temperature to each well and by removing the PBS by aspiration. PBS 1% BSA solution (2 mL) is added to each well to block non-specific sites and the plates are incubated for 1 hour at 37° C. in CO₂ incubator. The blocking solution is removed by aspiration and the wells are washed before adding the cells to the wells.

C. Addition of Cells

Before adding the cells (AC4 +/−and AC6 −/−) to the prepared plates, They are washed and removed from 10 cm culture dishes by incubating them for 10 minutes at 37° C. in the CO₂ incubator with 1.5 mL of trypsin/EDTA (0.05% trypsin, 0.53 mM EDTA) (GibcoBRL, Cat: 25300-054) solution. Detached cells are suspended in 5 mL of PBS at ambient temperature, placed in 15 mL conical tubes and centrifuged at 600 g on a clinical centrifuge for 5 minutes. PBS is removed by aspiration, then the cells are counted using a hemacytometer and cell concentration is adjusted to 1×10⁶ cells/mL in DMEM 0.5% BSA.

The cell suspension mixed with a test compound in an amount adequate to provide a range of 25 to 50 μM concentration is incubated for 30 minutes at 37° C. in the CO₂ incubator with mixing every. ten minutes. An aliquot is retained as a control to determine the basal phosphorylation level before fibronectin-treatment. For fibronectin treatment, 3 mL of the cell suspension is added to the fibronectin matrix in order to obtain 60% confluence (3×10⁶ cells/well) before incubating for 45 minutes at 37° C. in CO₂ incubator. Each sample is performed in duplicate.

At the end of fibronectin stimulation or incubation in suspension, cells are washed with ice-cold PBS supplemented with 1 mM sodium orthovanadate. Cells are lysed directly on the plate by adding 0.5 mL of ice-cold RIPA buffer supplemented with protease inhibitors and 1 mM sodium vanadate. Plates are incubated at 4° C. with frequent agitation for 10 minutes, then disrupted by repeated aspiration with a P1000™ micropipette before transfer to 1.5 mL microcentrifuge tubes. Cellular debris is pelleted at 13,000 rpm (10000 g) for 10 minutes at 4° C. in a microcentrifuge, and supernatants are drawn off into fresh 1.5 mL microcentrifuge tubes

Protein concentration in the cell lysates is assayed using Bio-Rad protein concentration kit DC™ (Bio-Rad) according to manufacturer's instructions. Immunoprecipitation of p130^{cas} is performed with an amount of 250 mg protein adjusted in a final volume of 1 mL with RIPA buffer supplemented with 1 mM vanadate and inhibitors.

For the immunoprecipitation, 1 mg (4 mL) of anti-p130^{cas} mouse monoclonal (Transduction Laboratories, Cat: P27820) is added to each sample and the mixture is incubated for 2 hours at 4° C. on a rotating device. As an immunoprecipitation control, the same amount of cell lysate is incubated at this step with 1 mg (3 mL) of rabbit pre-immune serum. Then 20 mL of resuspended Protein G-Agarose beads (GibcoBRL, Cat: 15920-010) is added and the mixture is incubated with agitation for 1 hour at 4° C. on a rotating device. Immunoprecipitates are collected by centrifugation at 2000 g for 5 minutes at 4° C. Pellets are washed 3 times with 1 mL of ice-cold RIPA buffer (the supernatant is removed by aspiration). After final wash, the beads are resuspended into 60 mL of SDS sample buffer.

D. SDS-PAGE and Western Blotting

30 μl of immunoprecipitate are separated on a 10% polyacrylamide gel for 1.5 hours at 125V (p130^{cas} is a 130kDa protein)

Briefly, nitrocellulose membranes are blocked with TBS-Tween (TBST): 20 mM Tris-HCl, pH 7.2-7.4 (BioShop, Cat#: TRS 001), 150 mM NaCl (BioShop, Cat#: SOD 001) and 0.1% (v/v) Tween-20 (BioShop, Cat: TWN508) 1% BSA for 1 hour with agitation at ambient temperature. Antiphosphotyrosine monoclonal antibody clone 4G10™ (Upstate Biotechnologies) is used at a 1/1000 dilution in TBST 1%BSA and incubated for 1 hour with agitation at ambient temperature. The anti-mouse-lgG-horseradish peroxidase (hrp) conjugate (Jackson Laboratories) is used at a 1/20,000 dilution in TBST 1%BSA and incubated for 1 hour at ambient temperature.

E. Data Analysis

The data are analyzed as a function of p130^{cas} phosphorylation status.

Compounds of the invention tested demonstrate a higher level of phosphorylation in the PTPN12 −/− cells when compared to the PTPN12 +/− cells after fibronectin-treatment. Inhibition of PTPN12 in the +/− cells by a compound of the invention results in a higher phosphorylation state of p130^{cas} in the treated cells when compared to the non-treated cells.

The foregoing assay is also used, with the appropriate starting reagents and enzyme preparations, to test the ability of the compounds of the invention to inhibit PTPN 12 activity.

### Example 6

### Cell Proliferation

This procedure (Jelinkova, R. B. et al., “Antiproliferative effect of a lectin- and anti-Thy-1.2 antibody-targeted HPMA copolymer-bound doxorubicin on primary and metastatic human colorectal carcinoma and on human colorectal carcinoma transfected with the mouse Thy-1.2 gene”, Bioconjug. Chem. (2000), Vol. 11, No. 5, pp. 664-73) is used to assess the effect compounds have on various cell lines with respect to proliferation. The rate of anchorage-independent growth of various tumor cells is quantified by measuring the amount of free isotopic thymidine that has been incorporated into the cells over a period of time. The effect of any compound to inhibit the proliferation of various tumor cells could be used as an indication of its ability to prevent disease progression in cancer.

Cultured tumour cells are harvested cells as per normal procedures: i.e. trypsinize, centrifuge and count cells. A volume of 90 μL is used to seed 5,000 cells/well in a 96 well plate. Cells are incubated for 24 hours at 37° C. under 5% CO₂. After incubation, cells should be 80-90% confluent.

³H-thymidine (Amersham) is diluted in cell culture media to a concentration of 100 μCi/mL. The test compound is diluted in the thymidine broth to 10× the final desired concentration.

Then 10 μL of diluted compound is added to the 90 μL of cells already present in the 96-well plates. Six replicates wells are done per treatment in columns 2 to 11. Plates were mixed by rocking.

A known cytotoxic compound such as staurosporine is used in relatively high concentrations as a positive control in column 1. Diluted DMSO is used as a negative control in column 12. The plate is incubated exactly 24 hours at 37° C.

After incubation, plates are observed under the microscope for obvious cell death, abnormal cell shape, crystal formation of the compound, etc. Then 25 μL volume of cold 50% TCA is added slowly to the 100 μL volume already in each well, and incubated for 1-2 hours at 4° C. The plates are then washed 5× in tap water and allowed to dry completely (usually overnight) at ambient temperature. Finally, 100 μL of scintillation fluid is added to each well and the plates are counted in a Wallac 1450 Microbeta™ counter according to user manual instructions.

The amount of inhibition is determined by the following formula:
% inhibition=100−[(AVG treatment−AVG positive control)/100(AVG negative control −AVG positive control)]

### Example 7

### Cytotoxicity Assay

This procedure is used to assess the effects compounds have on various cell lines with respect to cell viability. Cell viability is quantified using calcein AM and measuring its conversion to a fluorescent product (calcein) with a fluorimeter.

The principle of this assay is based on the presence of ubiquitous intracellular esterase activity found in live cells. By enzymatic reaction of esterase, non-fluorescent cell-permeant calcein AM is converted to the intensely fluorescent calcein. The polyanionic dye calcein is retained within live cells, producing a green fluorescence in live cells. It is a faster, safer, and better-correlated indicator of cytotoxicity than alternative methods (e.g. ³H-Thymidine incorporation). It should be noted that calcein AM is susceptible to hydrolysis when exposed to moisture. Therefore, prepare aqueous working solutions containing calcein AM immediately prior to use, and use within about one day.

A kit available to do this assay is “LIVE/DEAD® Viability/Cytotoxicity Kit (L-3224)” by Molecular Probes.

Cells were collected from tissue culture flasks and trypsinized, centrifuged, resuspended and counted. Cells were seeded to obtain 80-90% confluence (for normal cells, 10,000 cells/well (8000 cells/well for HUVEC cells)). A cell concentration of 110,000 cells/mL (88,000 cells/well for HUVEC cells) is prepared as 90 μL volume is used per well.

Using an 8-channel multi-dispense pipettor, cells were seeded in the central rows of the plate (Nunclon™ 96 well flat-bottom plate), leaving the peripheral top and bottom rows with same volume of media only. The plates were incubated at 37° C., 5% CO₂ overnight for approximately 24 hours.

For test compounds, cell culture media (e.g., RPMI+10%FBS), 10×compound solution of final desired concentration from 20 mM stock compounds was prepared.

10 μl of this 10× compound solution is added to the 90 μL of cells already present in the 96 well plates and a known cytotoxic compound from previous testing is used as a positive control. The negative control is 100% DMSO diluted to the same factor as the compounds.

The plates are incubated at 37° C. for approximately 24 hours, and media is aspirated after plates are spun at 2400 rpm for 10 min at ambient temperature. 100 μL of 1× DPBS (without calcium chloride, without magnesium chloride (GibcoBRL, cat#14190-144)) is added to each well.

The calcein AM solution is prepared by added 50 μg of calcein AM crystal (m.w. =994.87 g/mol, Molecular Probes, Eugene, Oreg.) and Anhydrous DMSO (Sigma Aldrich) to make 1 mM stock and diluting stock to 2× the final desired concentration in 1× DPBS just before the assay. 100 μL of this 2× was added to the 100 μL of DPBS in the wells and the plates are incubated at ambient temperature for 30 minutes. Fluorescence data was read and recorded (Fluoroskan Ascent® FL fluorimeter (excitation˜485nm, emission˜527nm)).

The values for replicates (usually six) are averaged and % inhibition is calculated as follows:
% inhibition=100−[(AVG treatment−AVG positive control)/(AVG negative control−AVG positive control)*100]
The compound 5-Benzylsulfanyl-3-methylsulfanyl-isothiazole-4-carboxylic acid amide showed no cytotoxicity in this assay.

### Example 8

To test the efficacy of test compounds on H460 subcutaneous xenograft alone and in combination with doxorubicin.

Athymic nude female mice are used for this experiment. A group of 60 mice are inoculated with five million H460 cells in 100 μL Matrigel™(VWR Canada) excipient. Tumours are measured three times a week with digital calipers and the tumour volumes calculated. When tumours have reached an average size of 100 mm³, about two weeks after tumour implantation. At that time any nongrowing ‘outliers’ are removed so that animals can be distributed into groupings that are equal and statistically the same tumour mass, i.e. divided into six groups with about 10 mice per group.

Treatments with test compounds continue for about 20 days, and will be oral (gavage), intravenous, subcutaneous, or intraperitoneal depending on the known solubility of the test compound. A dose of 25 mg/kg is typical for such testing, but the dose selected will reflect the potency of the compound and the route of administration. Up to 200 mg/kg may be selected.

Positive controls may alternately be doxorubicin or cisplatin, or cyclophosphamide.

At study termination, the mice are anesthetized 3 hours after the last dose of test compound, and plasma and tissues are harvested and frozen. Tumours are divided into the desired number of aliquots and fast frozen for later analysis.

### Example 9

### Cell Invasion in Matrigel™

This procedure is used to assess the compound effect on the tumor cell invasion through Matrigel™-coated Fluoroblok™ inserts. Invasion allows tumor cells to spread to sites other that the primary tumor. BD Bioscience's BioCoat™ FluoroBlok™ Invasion Systems combine the benefits of the BD BioCoat™ Matrigel™ Invasion Chambers with the fluorescence blocking membrane capabilities of the BD Falcon™ HTS FluoroBlok™ 24-Multiwell Insert System™. The following assay uses this system to assess compound effects on the anti-tumor cell invasion through layer of Matrigel™ extracellular matrix.

The cell lines used are HT 1080 (ATCC, Cat# CCL - 121), DU-145 (ATCC, Cat# HTB-81), PC3 (ATCC, Cat# CRL-1435) or B16F1 (ATCC, Cat# CRL-6323).

The invasion test system is removed from the package from −20° C. storage and allowed to warm to ambient temperature. PBS is added to the interior of the inserts and they are allowed to rehydrate for 2 hours at 37° C. Then the medium is removed and 450 μL cell suspensions of tumor cells (grown to 50-70% confluence, trypsinized, and resuspended in medium without serum at 1×10⁶/mL) is added to the top chamber. Test compounds are added to the top chamber at 10× the desired final concentration in 50 μL volumes. DMSO acts as the control.

Then 750 μL of medium containing 50% fresh growth medium with 10% FBS and 50% NIH 3T3-conditioned medium is added to each of the bottom wells. The invasion system is then incubated for 24 to 48 hours at 37° C., in a 5% CO₂ atmosphere.

Following incubation, the insert plate is transferred into a second 24-well plate containing 0.5 mL of 5 μg/mL calcein AM (Molecular Probes) in Hanks Buffered Salt Solution (HBSS), and plates are incubated for 1 hour at 37° C., 5% CO₂.

Fluorescence data indicating cell invasion is read in a Fluoroskan Ascent™ FL (LabSystems) with bottom reading at excitation/emission wavelength of 485/538 nm.

Data is expressed as fluorescence units (FU) from the sum of middle 25 areas per 24-well or as percentage of invasion inhibition by following formula: % of invasion inhibition=100−FU of compound treated cell invasion/FU of DMSO treated cell invasion*100.

The compounds inhibit invasion in this assay, and thus may be used to prevent metastasis in cancer and tissue remodeling.

### Example 10

### Peritoneal Macrophage Stimulation and Analysis

A. Establishment of inflammation assay panel.

Macrophages are important elements of innate immunity to infection and are among the first cell type in the immune response to be exposed to and activated by infectious agents. IFN-γ and LPS are potent activators of macrophages, priming them for a variety of biological effects. IFN-γ, initially secreted by NK and T cells in response to infection, converts macrophages from a resting to an activated state (inflammatory macrophages), priming them for antimicrobial activity manifested by increased killing of intracellular pathogens, and antigen processing and presentation to lymphocytes. The action of IFN-γ is synergized with the LPS second messenger, enhancing the stimulation of macrophages through the activation of NF-κB, that results in the transcriptional up-regulation of a number of genes involved in the cell-mediated immune response, including inducible nitric oxide synthase (iNOS). Activated macrophages are qualitatively different from quiescent macrophages. These differences are typically observed by an increased proliferation index, up-regulated expression of MHC-II, and production of various bioactive molecules. The latter biological effects are mediated by nitric oxide (NO) release and increased production of pro-inflammatory cytokines (IL-6, TNF-γ, IL-1). Primary macrophages derived from Balb/c and RAW 264.7 cells (Balb/c background) were used to establish in vitro inflammatory models with fast and reliable readouts.

B. Materials and Methods

1. Reagents.

The iNOS inhibitor NG-Monomethyl-L-arginine (L-NMMA) and murine rIFN-γ were purchased from Calbiochem, (San Diego, Calif.). Protein-free, phenol/water-extracted LPS (from E. coli serotype 0111 :B4 0127:B8), Zymosan A, dexamethasone and hydrocortisone, sulfanilamide and N-(1-naphthyl)ethylenediamine, were purchased from Sigma (St. Louis, Mo.). Human recombinant vascular endothelial growth factor (VEGF) was purchased from R&D Systems (Minneapolis, Minn.). Rabbit polyclonal antibody against active (phosphorylated) extracellular signal-regulated kinase (ERK), as well as horse radish peroxidase (HRP)-conjugated donkey anti-rabbit IgG were obtained from Promega (Madison, Wis.). ELISA dual-set kit for detection of IL-6 was purchased from PharMingen (San Diego, Calif.). Anti-murine iNOS/NOS type II and cyclooxygenase 2 (COX-2) antibodies were obtained from Transduction Laboratories (Lexington, Ky.).

Female, 6-12 wk of age, BALB/c mice were purchased from Harlan Inc. (Indianapolis, IN) and housed under fluorescent light for 12 h per day. Mice are housed and maintained in compliance with the Canadian Council on Animal Care standards.

2. Isolation of primary mouse macrophages.

Peritoneal exudate macrophages were isolated by peritoneal lavage with ice-cold sterile physiological saline 24 hours after intraperitoneal injection of BALB/c mice with 0.5 mL of sterile Zymosan A (1 mg/0.5 mL 0.9% saline). Cells were washed, resuspended in RPMI 1640 supplemented with 1 mM D-glucose, 1mM sodium pyrovate, 100 units/mL penicillin, 100 μg/mL streptomycin, and 5% FBS.

3. Treatment of primary macrophages.

Primary macrophages (1.5×10⁵ cells/well) were grown in 96-well plates (nitrite assay), or 6-well plates (2×10⁶ cells/well) for measurement of iNOS and COX-2 expression. Following 3 hours incubation, at 37° C., 5% CO₂ (allowing macrophages to attach) cells were stimulated with LPS (5 μg/mL) and IFN-γ (100 U/mL) in the absence or presence of various concentrations of test compounds (all treatments were replicated six times). Cells were incubated for an additional 24 hours, and cell free culture supernatants from each well were collected for NO and cytokine determination. The remaining cells were stained with crystal violet or MTS to determine effect of the test compounds on cell survival.

4. NO production.

Following stimulation, the production of NO was determined by assaying culture supernatants for NO₂⁻, a stable reaction product of NO with molecular oxygen. Briefly, 100 μL of culture supernatant was reacted with an equal volume of Griess reagent at ambient temperature for 10 minutes. The absorbance at 550 nm was determined. All measurements were performed six times. The concentration of NO₂⁻was calculated by comparison with a standard curve prepared using NaNO₂.

5. Western blot analysis.

After incubation with the indicated stimuli in the presence of inhibitors, cells (duplicate samples, 2×10⁶cell/6-wells plate) were washed in PBS and lysed on ice in 60 μL of lysis buffer. The protein content of each sample was determined using the Bradford protein assay kit (Bio-Rad, Richmond, Calif.). Absorbance was measured at 750 nm with a Beckman DU530 spectrophotometer (Palo Alto, Calif.). Proteins were mixed with 45×SDS sample buffer. Following separation of proteins by SDS-PAGE, using 8% bis-acrylamide in the separation gel, the proteins were transferred from the gels onto PVDF membranes using a MiniProtean™ III Cell (Bio-Rad), at 100 V for 1.5 hours. Equal amounts of protein (5 μg) were loaded onto SDS-PAGE gels and examined by Western blot analysis with anti-Actin, anti-iNOS, anti-COX-2 murine monoclonal antibodies, according to the manufacturer's specifications (Transduction Laboratories). Primary antibodies, in 5% blocking buffer (5% NFM/TTBS), were incubated with blots 2 hours at RT or overnight at 4° C., followed by incubation with peroxidase-conjugated secondary antibody. Chemiluminescence substrates were used to reveal positive bands. The bands were exposed on X-ray films. The films are used to analyze the impact of inhibitors on expression of iNOS and Cox-2 compared to various controls and “house-keeping” protein (actin) concentration to control the protein loading and detect any non-specific effects on protein production. The Multi-Analyst™/PC system from Biorad was used to quantitate the bands of the expressed protein on the film. This version of Multi-Analyst™ is used with the Bio-Rad Gel Doc 1000™ imaging system. White light is chosen as the selected light source, thus the signal strength is measured in OD (optic density) units. The OD of each band is being subtracted to arrive at a global background area of the gel.

C. In Vitro Angiogenesis.

HUVEC cells cultured for 24 hours in M199 with 0.5% FCS were plated at 6×10⁵ cells/well in 12-well plates pre-coated with 300 μL of Matrigel™ (10.7 mg/mL; Becton Dickinson) in M199 with 0.5% FCS in the presence of VEGF (1ng/mL), and in the absence or presence of positive control (Z)-3-[2,4-dimethyl-5-(2-oxo-1,2-dihydroindol-3-ylidenemethyl)1H-pyrrol-3-yl]propionic acid or various inhibitors. After 5 hours of incubation in a 5% CO₂-humidified atmosphere at 37° C., the three-dimensional organization of the cells was examined using an inverted photomicroscope. The cells were fixed with crystal violet (0.05% in 20% ethanol) and digitally photographed.

C. Enzyme immunoassays for mouse IL-6.

IL-6 levels were determined with PharMingen's OptEIA™ ELISA set developed using an anti-mouse IL-6 antibody pair and mouse rIL-6 standard (PharMingen). Maxisorp™ F16 multiwell strips (Nunc, Roskilde, Denmark) were coated with anti-mouse IL-6 capture antibody (at recommended concentration) in 0.1 M NaHCO₃, pH 9.5, 100 μL/well, overnight at 4° C. Plates were washed three times with 0.05% Tween 20 in PBS (PBST) and blocked for 1 hour at ambient temperature with 200 μL/well of 10% FCS in PBS (blocking and dilution buffer). Plates were washed three times with PBST and duplicate samples (100 μL/well) or standards (100 μL/well) in diluent buffer were incubated for 2 hours at ambient temperature. Plates were washed five times with PBST and incubated with biotinylated anti-mouse IL-6 and avidin-HRP conjugate (at concentrations recommended by the manufacturer) for 1 hour at ambient temperature. Plates were washed seven times with PBST and 100 μL of 3,3′5,5′ tetramethylbenzidine substrate solution (TMB substrate reagent set, BD PharMingen) was added to each well. After 15-30 minute incubation at ambient temperature, colour development was terminated by adding 50 μL of 2 N H₂SO₄ (Sigma). Absorbance was read at 450 nm with an EL 312e™ microplate reader (or equivalent). The lower limit of detection for IL-6 was 15.6 pg/mL.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-14. (canceled)

15. A pharmaceutical composition useful in treating cancer or inflammation in a human, wherein the pharmaceutical composition comprises a pharmaceutically acceptable carrier, diluent or excipient and a compound of formula (II): 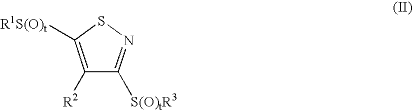
wherein:
each t is independently 0, 1 or 2;
R¹ and R³ are each independently alkyl, alkenyl, aryl, aralkyl, aralkenyl, cycloalkyl, cycloalkylalkyl, cycloalkylalkenyl, haloalkyl, haloalkenyl, haloalkoxyalkyl, haloalkoxyalkenyl, —R⁴—N═N—O—R⁵, —N(R⁶)₂ or heterocyclylalkyl;
R² is hydrogen, alkyl, alkenyl, aryl, aralkyl, aralkenyl, cycloalkyl, cycloalkylalkyl, cycloalkylalkenyl, halo, haloalkyl, haloalkenyl, nitro, —R⁴—N═N—O—R⁵, —OR⁶, —C(O)OR⁶, —N(R⁶)₂, —C(O)N(R⁶)_{2,} —N(R⁶)C(O)OR⁵, —N(R⁶) C(O)N(R⁶)₂, heterocyclyl or heterocyclylalkyl;
R⁴ is a bond or a straight or branched alkylene or alkenylene chain;
each R⁵ is independently hydrogen, alkyl, alkenyl, haloalkyl, haloalkenyl, aralkyl, aralkenyl, cycloalkyl, cycloalkylalkyl or cycloalkylalkenyl; and
each R⁶ is independently hydrogen, alkyl, alkenyl, haloalkyl, haloalkenyl, aryl, aralkyl, aralkenyl, cycloalkyl, cycloalkylalkyl or cycloalkylalkenyl;
provided that when t is 0 and R¹ and R³ are both methyl, R² can not be —C(O)OH, —C(O)NH₂, carboxymethyl or unsubstituted phenyl;
as a single stereoisomer, a mixture of stereoisomers, or as a racemic mixture of stereoisomers; or as a solvate or polymorph; or as a pharmaceutically acceptable salt thereof.

16. The pharmaceutical composition of claim 15 wherein the R¹ substituent of the compound of formula (II) is alkyl or alkenyl.

17. The pharmaceutical composition of claim 15 wherein the R¹ substituent of the compound formula (II) is aryl, aralkyl or aralkenyl.

18. The pharmaceutical composition of claim 15 wherein the R¹ substituent of the compound formula (II) is cycloalkyl, cycloalkylalkyl or cycloalkylalkenyl.

19. The pharmaceutical composition of claim 15 wherein the R¹ substituent of the compound of formula (II) is haloalkyl, haloalkenyl, haloalkoxyalkyl or haloalkoxyalkenyl.

20. The pharmaceutical composition of claim 15 wherein the R¹ substituent of the compound of of formula (II) is —R⁴—N═N—O—R⁵.

21. The pharmaceutical composition of claim 15 wherein the R¹ substituent of the compound of of formula (II) is —N(R⁶)2.

22. The pharmaceutical composition of claim 15 wherein the R¹ substituent of the compound of formula (II) is heterocyclylalkyl.

23. The pharmaceutical composition of claim 15 wherein the R² substituent of the compound of formula (II) is hydrogen, alkyl or alkenyl.

24. The pharmaceutical composition of claim 15 wherein the R² substituent of the compound of formula (II) is aryl, aralkyl or aralkenyl.

25. The pharmaceutical composition of claim 15 wherein the R² substituent of the compound of formula (II) is cycloalkyl, cycloalkylalkyl or cycloalkylalkenyl.

26. The pharmaceutical composition of claim 15 wherein the R² substituent of the compound of formula (II) is halo, haloalkyl or haloalkenyl.

27. The pharmaceutical composition of claim 15 wherein the R² substituent of the compound of formula (II) is nitro or —R⁴—N═N—O—R⁵.

28. The pharmaceutical composition of claim 15 wherein the R² substituent of the compound of formula (II) is —OR⁶.

29. The pharmaceutical composition of claim 15 wherein the R² substituent of the compound of formula (II) is —C(O)OR⁶.

30. The pharmaceutical composition of claim 15 wherein the R² substituent of the compound of formula (II) is —N(R⁶)₂.

31. The pharmaceutical composition of claim 15 wherein the R² substituent of the compound of formula (II) is —C(O)N(R⁶)₂ or —N(R⁶)C(O)OR⁵.

32. The pharmaceutical composition of claim 15 wherein the R² substituent of the compound of formula (II) is heterocyclyl or heterocyclylalkyl.

33. The pharmaceutical composition of claim 15 wherein t is 0.

34. The pharmaceutical composition of claim 15 wherein t is 1.

35. The pharmaceutical composition of claim 15 wherein t is 2.

36. A method of treating cancer, inflammation or a hyperproliferative disorder in a mammal, which method comprises administering to the mammal in need thereof a therapeutically effective amount of a compound of formula (I): 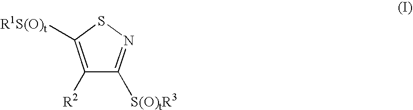
wherein:
each t is independently 0, 1 or 2;
R¹ and R³ are each independently alkyl, alkenyl, aryl, aralkyl, aralkenyl, cycloalkyl, cycloalkylalkyl, cycloalkylalkenyl, haloalkyl, haloalkenyl, haloalkoxyalkyl, haloalkoxyalkenyl, —R⁴—N═N—O—R⁵, —N(R⁶)₂ or heterocyclylalkyl;
R² is hydrogen, alkyl, alkenyl, aryl, aralkyl, aralkenyl, cycloalkyl, cycloalkylalkyl, cycloalkylalkenyl, halo, haloalkyl, haloalkenyl, cyano, nitro, —R⁴—N═N—O—R⁵, —OR⁶, —C(O)OR⁶, —N(R⁶)₂, —C(O)N(R⁶)₂,—N(R ⁶)C(O)OR⁵, —N(R⁶)C(O)N(R⁶)₂, heterocyclyl or heterocyclylalkyl;
R⁴ is a bond or a straight or branched alkylene or alkenylene chain;
each R⁵ is independently hydrogen, alkyl, alkenyl, haloalkyl, haloalkenyl, aralkyl, aralkenyl, cycloalkyl, cycloalkylalkyl or cycloalkylalkenyl; and
each R⁶ is independently hydrogen, alkyl, alkenyl, haloalkyl, haloalkenyl, aryl, aralkyl, aralkenyl, cycloalkyl, cycloalkylalkyl or cycloalkylalkenyl;
as a single stereoisomer, a mixture of stereoisomers, or as a racemic mixture of stereoisomers; or as a solvate or polymorph; or as a pharmaceutically acceptable salt thereof.

37. (canceled)

38. The method according to claim 36 wherein the cancer, inflammation or hyperproliferative disorder is associated with tissue remodelling or repair.

39. The method according to claim 36 wherein the cancers, inflammation or hyperproliferative disorder is associated with the activity of PTPN12.

40. (canceled)

41. A method of treating a mammal having a disorder or condition associated with hyperproliferation and tissue remodelling or repair, wherein said method comprises administering to the mammal having the disorder or condition a therapeutically effective amount of a compound of formula (I): 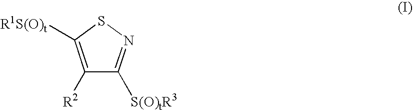
wherein:
each t is independently 0, 1 or 2;
R¹ and R³ are each independently alkyl, alkenyl, aryl, aralkyl, aralkenyl, cycloalkyl, cycloalkylalkyl, cycloalkylalkenyl, haloalkyl, haloalkenyl, haloalkoxyalkyl, haloalkoxyalkenyl, —R⁴—N═N—O—R⁵, —N(R⁶)₂ or heterocyclylalkyl;
R² is hydrogen, alkyl, alkenyl, aryl, aralkyl, aralkenyl, cycloalkyl, cycloalkylalkyl, cycloalkylalkenyl, halo, haloalkyl, haloalkenyl, cyano, nitro, —R⁴—N═N—O—R⁵, —OR⁶, —C(O)OR⁶, —N(R⁶)₂, —C(O)N(R⁶)₂, —N(R ⁶)C(O)OR⁵, —N(R⁶)C(O)N(R⁶)₂, heterocyclyl or heterocyclylalkyl;
R⁴ is a bond or a straight or branched alkylene or alkenylene chain;
each R⁵ is independently hydrogen, alkyl, alkenyl, haloalkyl, haloalkenyl, aralkyl, aralkenyl, cycloalkyl, cycloalkylalkyl or cycloalkylalkenyl; and
each R⁶ is independently hydrogen, alkyl, alkenyl, haloalkyl, haloalkenyl, aryl, aralkyl, aralkenyl, cycloalkyl, cycloalkylalkyl or cycloalkylalkenyl;
as a single stereoisomer, a mixture of stereoisomers, or as a racemic mixture of stereoisomers; or as a solvate or polymorph; or as a pharmaceutically acceptable salt thereof.

42. The method according to claim 41 wherein the mammal is a human.

43. A method of treating a mammalian cell with a compound of formula (I): 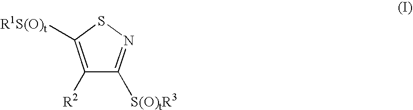
wherein:
each t is independently 0, 1 or 2;
R¹ and R³ are each independently alkyl, alkenyl, aryl, aralkyl, aralkenyl, cycloalkyl, cycloalkylalkyl, cycloalkylalkenyl, haloalkyl, haloalkenyl, haloalkoxyalkyl, haloalkoxyalkenyl, —R⁴—N═N—O—R⁵, —N(R⁶)₂ or heterocyclylalkyl;
R² is hydrogen, alkyl alkenyl, aryl, aralkyl, aralkenyl, cycloalkyl, cycloalkylalkyl, cycloalkylalkenyl, halo, haloalkyl, haloalkenyl, cyano, nitro, —R⁴—N═N—O—R⁵, —OR⁶, —C(O)OR⁶, —N(R⁶)₂, —C(O)N(R⁶)_{2, —N(R} ⁶)C(O)OR⁵, —N(R⁶)C(O)N(R⁶)₂, heterocyclyl or heterocyclylalkyl;
R⁴ is a bond or a straight or branched alkylene or alkenylene chain;
each R⁵ is independently hydrogen, alkyl, alkenyl, haloalkyl, haloalkenyl, aralkyl, aralkenyl, cycloalkyl, cycloalkylalkyl or cycloalkylalkenyl; and
each R⁶ is independently hydrogen, alkyl, alkenyl, haloalkyl, haloalkenyl, aryl, aralkyl, aralkenyl, cycloalkyl, cycloalkylalkyl or cycloalkylalkenyl;
as a single stereoisomer, a mixture of stereoisomers, or as a racemic mixture of stereoisomers; or as a solvate or polymorph; or as a pharmaceutically acceptable salt thereof,
wherein the method comprises administering the compound of formula (I) to a mammalian cell and the compound of formula (I) is capable of inhibiting the activity of PTPN12 within the mammalian cell.

44. The method of claim 43 wherein the mammalian cell is treated in vitro.

45. The method of claim 43 wherein the mammalian cell is treated in vivo.

46. The method of claim 43 wherein the inhibition of activity results in a reduction of cell adhesion.

47. The method of claim 43 wherein the inhibition of activity results in a reduction of cell division.

48. The method of claim 43, wherein the inhibition of activity results in a reduction of cell migration.

49. The method of claim 43, wherein the inhibition of activity results in control of tumor growth.

50. The method of claim 43 wherein the inhibition of activity results in control of lymphocyte activation.

51. The method of claim 36 wherein the R¹ substituent of the compound of formula (I) is alkyl or alkenyl.

52. The method of claim 36 wherein the R¹ substituent of the compound of formula (I) is aryl, aralkyl or aralkenyl.

53. The method of claim 36 wherein the R¹ substituent of the compound of formula (I) is cycloalkyl, cycloalkylalkyl or cycloalkylalkenyl.

54. The method of claim 36 wherein the R¹ substituent of the compound of formula (I) is haloalkyl, haloalkenyl, haloalkoxyalkyl or haloalkoxyalkenyl.

55. The method of claim 36 wherein the R¹ substituent of the compound of formula (I) is —R⁴—N═N—O—R⁵.

56. The method of claim 36 wherein the R¹ substituent of the compound of formula (I) is —N(R⁶)₂.

57. The method of claim 36 wherein the R¹ substituent of the compound of formula (I) is heterocyclylalkyl.

58. The method of claim 36 wherein the R² substituent of the compound of formula (I) is hydrogen, alkyl or alkenyl.

59. The method of claim 36 wherein the R² substituent of the compound of formula (I) is aryl, aralkyl or aralkenyl.

60. The method of claim 36 wherein the R² substituent of the compound of formula (I) is cycloalkyl, cycloalkylalkyl or cycloalkylalkenyl.

61. The method of claim 36 wherein the R² substituent of the compound of formula (I) is halo, haloalkyl or haloalkenyl.

62. The method of claim 36 wherein the R² substituent of the compound of formula (I) is nitro or —R⁴—N═N—O—R⁵.

63. The method of claim 36 wherein the R² substituent of the compound of formula (I) is —OR⁶.

64. The method of claim 36 wherein the R² substituent of the compound of formula (I) is —C(O)OR⁶.

65. The method of claim 36 wherein the R² substituent of the compound of formula (I) is —N(R ⁶)₂.

66. The method of claim 36 wherein the R² substituent of the compound of formula (I) is —C(O)N(R⁶)₂ or —N(R⁶)C(O)OR⁵.

67. The method of claim 36 wherein the R² substituent of the compound of formula (I) is heterocyclyl or heterocyclylalkyl.

68. The method of claim 36 wherein t is 0.

69. The method of claim 36 wherein t is 1.

70. The method of claim 36 wherein t is 2.
